(19) United States
(12) Patent Application Publication
(10) Pub. No.: US 2002/0183277 A1
(43) Pub. Date: Dec. 5, 2002

(54) Combination of vitamin D analogue and pyrimidine nucleoside analogue

(76) Inventor: Lise Binderup, Taastrup (DK)

Correspondence Address: BIRCH STEWART KOLASCH & BIRCH, PO BOX 747, FALLS CHURCH, VA, 22040-0747, US

(21) Appl. No.: 10/142,778

(22) Filed: May 13, 2002

(30) Foreign Application Priority Data
May 15, 2001 (DK) PA 2001 00759

(51) Int. Cl. A61K 31/7072; A61K 31/59; A61K 31/513

(52) U.S. Cl. 514/50; 514/167; 514/269

(57) ABSTRACT

The present invention relates to a pharmaceutical composition comprising a vitamin D analogue capable of upregulating the expression of cytidine deaminase in tumour cells and a cytostatic pyrimidine nucleoside analogue and its use in a combination treatment of neoplastic diseases.

### FIELD OF INVENTION

The present invention relates to a composition comprising a vitamin D analogue and a cytostatic pyrimidine nucleoside analogue, as well as a combination treatment of neoplastic diseases by administration of a vitamin D analogue and a cytostatic pyrimidine analogue.

### BACKGROUND OF THE INVENTION

Fluorouracil (5-fluorouracil, 5-FU) has been extensively used for more than 30 years in the treatment of solid tumours, in particular gastrointestinal, breast and head and neck cancers. 5-FU acts by forming a complex with thymidylate synthase, thereby preventing the formation of deoxythymidine monophosphate from deoxyuridine monophosphate and decreasing the availability of deoxythymidine triphosphate for DNA replication and repair. Inhibition of thymidine synthase also gives rise to an increase of deoxyuridine monophosphate in the cell. The monophosphate may be anabolised to the triphosphate level and, in the form of deoxyuridine triphosphate (and fluorodeoxyuridine triphosphate), the latter can be incorporated into DNA, contributing to the inhibition of DNA elongation and altering DNA chain stability. However, 5-FU is in itself an unstable compound in that it is extensively catabolised on intravenous administration to the inactive form dihydro-5-FU within minutes of administration. Also, on oral administration, 5-FU is degraded in the gastrointestinal tract by dihydropyrimidine dehydrogenase so that oral bioavailability of the drug is erratic and incomplete. Treatment with 5-FU gives rise to severe cytotoxic effects, in particular on rapidly dividing tissue such as gastrointestinal mucosa and bone marrow, and the dose-limiting toxicity of 5-FU has resulted in a limited clinical efficacy of 5-FU (i.e. an overall response rate of 10-30%).

Efforts have therefore been made to develop derivatives of 5-FU which have an improved efficacy and lower systemic toxicity than 5-FU and which can be administered orally. In this context, it has been proposed to utilise a metabolic pathway involving, inter alia, the enzyme cytidine deaminase which catalyses the deamination of the natural pyrimidine metabolites deoxycytidine and cytidine to deoxyuridine and uridine, respectively. One interesting class of such 5-FU derivatives is fluoropyrimidine prodrugs which are converted by a carboxylesterase in the liver to 5′-deoxy-5-fluorocytidine. The metabolite is then further converted by cytidine deaminase to 5′-deoxy-5-fluorouridine which, in turn, is converted to 5-FU by thymidine phosphorylase. As levels of thymidine phosphorylase are increased in many tumours, the tumour concentrations of 5-FU following administration of fluoropyrimidine prodrugs are 20 times higher than in normal tissue (N. Damjanov and N. J. Meropol, Oncology 14(6), Jun. 2000, pp. 799-807).

A number of research groups have observed that the expression of cytidine deaminase in tumour cell lines may be modified by 1α, 25-dihydroxyvitamin D₃. Thus, J. N. Mejer et al., Leuk. Res. 12(15), 1988, pp. 405-409, and J. N. Mejer et al., Med. Oncol. & Tumor Pharmacother. 7(1), 1990, pp. 25-29, report the finding that 1α, 25-dihydroxyvitamin D₃ causes an increase of cytidine deaminase in the human promyeolytic cell line HL60, resulting in an inhibition of the growth inhibitory effect of 1-β-D-arabinofuranosyl-cytosine on the cells. S.-I. Watanabe and T. Ichida, Biochim. Biophys Acta 1312, 1996, pp. 99-104, report the finding that 1α, 25-dihydroxyvitamin D₃ upregulates the expression of the cytidine deaminase gene in HL60 cells (a myeloid leukemia cell line), WiDr, Colo 201, Colo 205, CXF 280 and DLD-1 cells (colorectal carcinoma cell lines), MKN 45, MKN 1 and MKN 28 cells (gastric carcinoma cell lines) and the lung adenocarcinoma cell line A549. The authors speculate that combination treatment with 1α, 25-dihydroxyvitamin D₃ or analogues could increase the efficacy of fluoropyrimidine prodrugs in some tumours. However, no experimental evidence is presented in support of this hypothesis.

On the other hand, H. Matsumoto et al., Oncology Reports 6, 1999, pp. 349-352, report on the effect of a vitamin D analogue, 22-oxacalcitriol, in the treatment of estrogen receptor-negative MDA-MB-231 tumours in athymic mice alone or in combination with doxifluridine (5′-deoxy-5-fluorouridine, an intermediate in the conversion of fluoropyrimidine prodrugs to 5-FU) which is also often used in the treatment of breast cancer. The authors conclude that the effect of the combination treatment did not exceed that of treatment with 22-oxacalcitriol alone.

### SUMMARY OF THE INVENTION

It has surprisingly been found that certain vitamin D analogues have a more potent effect on the upregulation of cytidine deaminase expression in tumour cell lines than 1α, 25-dihydroxyvitamin D₃ and that they may, in addition, give rise to an upregulation of thymidine phosphorylase in certain tumour cells. An object of the invention is therefore to provide a combination of such vitamin D analogues with pyrimidine nucleoside cytostatic agents, in particular fluoropyrimidine prodrugs, in order to provide a more efficient conversion of such prodrugs to active 5-FU in tumour cells.

Accordingly, in one aspect, the present invention relates to a pharmaceutical composition comprising, as a first active ingredient, a vitamin D analogue capable of upregulating the expression of cytidine deaminase in tumour cells and, as a second active ingredient, a cytostatic pyrimidine nucleoside analogue together with a pharmaceutically acceptable excipient or vehicle.

In another aspect, the invention relates to a pharmaceutical combination composition comprising, in separate containers and intended for simultaneous or sequential administration, a vitamin D analogue capable of upregulating the expression of cytidine deaminase in tumour cells as a first active ingredient together with a pharmaceutically acceptable excipient or vehicle and a cytostatic pyrimidine nucleoside analogue as a second active ingredient together with a pharmaceutically acceptable excipient or vehicle.

### BRIEF DESCRIPTION OF THE DRAWINGS

FIG. 1 is a graph showing the cytidine deaminase (CDA) activity in HT-29 cells upon treatment with isopropanol (control), 1α, 25-dihydroxyvitamin D₃ (1,25(OH)2D3) and seocalcitol.

FIG. 2 is a graph showing the thymidine phosphorylase (TP) activity in HT-29 cells upon treatment with isopropanol (control), 1α, 25-dihydroxyvitamin D₃ (1,25(OH)2D3) and seocalcitol.

FIG. 3 is a graph showing the growth of FHC cells in the presence of 1α, 25-dihydroxyvitamin D₃ (1,25(OH)₂D₃) and seocalcitol (EB 1089) compared to a control.

### DETAILED DESCRIPTION OF THE INVENTION

Vitamin D Analogue

In the present context, the term “vitamin D analogue” is intended to indicate a synthetic compound comprising a vitamin D scaffold with side chain modifications and/or modifications of the scaffold itself. The term is not intended to include naturally occurring vitamin D derivatives such as metabolites. For the present purpose, the vitamin D analogue is preferably one that exhibits an at least 50%, preferably at least 75%, e.g. at least 100%, higher cytidine deaminase expression upregulating activity in tumour cells compared to the activity of 1α, 25-dihydroxyvitamin D₃.

In another preferred embodiment, the vitamin D analogue is one which is capable of upregulating the expression of both cytidine deaminase and thymidine phosphorylase in tumour cells so as to effect increased levels of conversion of a fluoropyrimidine prodrug to 5-fluorouracil in tumour cells. It is expected that a more efficient conversion of the prodrug to 5-FU in tumour tissue will make it possible to administer lower doses of the prodrug, whereby the dose-limiting systemic toxicity encountered with the administration of fluoropyrimidine prodrugs may be significantly reduced, if not completely avoided.

It has advantageously been found that vitamin D analogues do not upregulate cytidine deaminase expression in normal (non-cancerous cells), cf. example 2 below, suggesting that the toxic effects of 5-FU generated by enzymatic conversion of a fluoropyrimidine prodrug are unlikely to emerge in normal cells but will most likely be restricted to tumour cells.

Useful vitamin D analogues for the present purpose may be found in the class of compounds of formula I

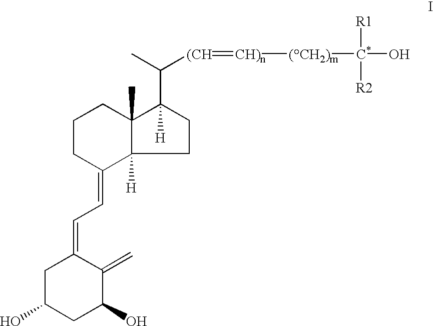

wherein

n is 2 or 3, m is 0 or an integer from 1 to 4;

R¹ and R², which are the same or different, are independently hydrogen or C₁₋₈ hydrocarbyl or, together with the carbon atom to which they are attached (marked with an asterisk in formula I), R¹ and R² form a saturated or unsaturated C₃₋₈ carbocyclic ring, R¹ and/or R ² and/or one of the m carbon atoms (marked with “^{o}” in formula I) being optionally substituted by one or more chloro or fluorine atoms or C₁₋₂ alkyl; or derivatives of compounds of formula I in which one or more hydroxy groups are transformed into —O-acyl or —O-glycosyl or phosphate ester groups, such masked groups being hydrolysable in vivo.

In the present context, the term “hydrocarbyl” is intended to indicate the residue left after removal of a hydrogen atoms from a straight, branched or cyclic, satyrated or unsaturated hydrocarbon. Compounds of formula I and methods of their preparation are described in WO 91/00855, the disclosure of which is hereby incorporated by reference in its entirety. One example of a suitable compound of formula I is seocalcitol.

Other useful vitamin D analogues for the present purpose may be found in the class of compounds of formula II

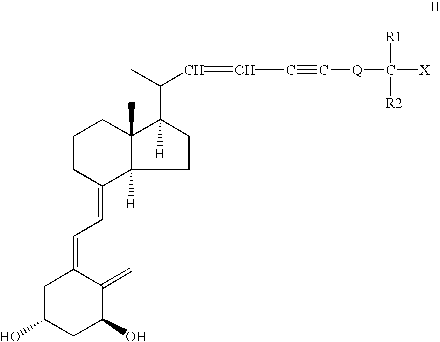

wherein,

X is hydrogen or hydroxy,

R¹ and R² which are the same or different independently represent hydrogen or C₁₋₄ hydrocarbyl, or, together with the carbon atom to which they are attached (marked with an asterisk in formula I), R¹ and R² form a saturated or unsaturated C₃₋₈ carbocyclic ring,

Q is a bond or a C₁₋₄ hydrocarbylene diradical,

R¹ and/or R² and/or Q being optionally substituted by one or more fluorine atoms; or prod rugs of compounds of formula II in which one or more of the hydroxy groups are masked as groups which can be converted to hydroxy groups in vivo.

In the present context, the term “hydrocarbylene diradical” is intended to indicate the residue left after removal of two hydrogen atoms from a straight, branched or cyclic, saturated or unsaturated hydrocarbon.

Compounds of formula II and methods of their preparation are described in WO 95/02577, the disclosure of which is hereby incorporated by reference in its entirety. One example of a suitable compound of formula II is one in which R¹ and R² are both ethyl, X is hydroxy and Q is a single bond.

Pyrimidine Nucleoside Analogues

In the present context, the term “cytostatic pyrimidine nucleoside analogue” is intended to indicate a synthetic analogue of a naturally occurring precursor of the pyrimidine synthesis pathway, the analogue exhibiting cytostatic or cytotoxic activity. Pyrimidine nucleoside analogues block incorporation of pyrimidines in DNA or RNA and may themselves be incorporated in DNA or RNA, resulting in the formation of defective DNA or RNA and ultimately cell death. The pyrimidine nucleoside analogues are most active in rapidly dividing tissue, including tumour tissue.

A number of pyrimidine nucleoside analogues are substrates for cytidine deaminase. Examples of such analogues which are currently used in the treatment of cancer are fluoropyrimidine prodrugs of 5-FU, as well as 1-β-D-arabinofuranosylcytosine (araC), doxifluridine and difluorodeoxycytidine (gemcitabine). In case of araC and gemcitabine, cytidine deaminase catalyses the initial degradation of these agents, whereas the enzyme catalyses the activation of 5-FU prodrugs to the active compound. Thus, by manipulating the cytidine deaminase activity in tumour cells, it may be possible to target the treatment of neoplastic diseases with various pyrimidine nucleoside analogues.

For the present purpose, it is preferred that the pyrimidine nucleoside analogue is a fluorouracil prodrug as these have been found to exhibit an improved oral bioavailability compared to 5-FU since they are not degraded by dehydropyrimidine dehydrogenase in the gastrointestinal tract. Examples of suitable fluoropyrimidine prodrugs are N⁴-pentoxycarbonyl-5′-deoxy-5-fluorocytidine (capecitabine, available from Hoffmann-La Roche under the trademark Xeloda™) and N⁴-trimethoxybenzoyl-5′-deoxy-5-fluorocytidine (galocitabine).

Pharmaceutical Compositions

Pharmaceutical compositions of the invention comprising a vitamin D analogue and a pyrimidine nucleoside analogue may be in unit dosage form such as tablets, pills, capsules, powders, granules, elixirs, syrups, emulsions, ampoules, suppositories or parenteral solutions or suspensions; for oral, parenteral or rectal administration or in any other manner appropriate for the formulation of cytostatic or cytotoxic compounds and in accordance with accepted practices such as those disclosed in Remington: The Science and Practice of Pharmacy. 20^{th} Ed. 2000.

It should be noted that the invention also includes within its scope embodiments where the vitamin D analogue and the pyrimidine nucleoside analogue are not combined in one and the same dosage form, but rather are provided in discrete unit dosage forms and administered separately (either substantially simultaneously or sequentially). When the active ingredients included in the present composition are not administered substantially simultaneously, the interval between dosing the individual active ingredients and the dosing frequency is determined by a variety of factors including, but not limited to, the severity of the condition to be treated, the age and general condition of the patient and progress of the treatment. It is within the capability of the skilled physician to determine the interval between the individual dosages as well as the dosage frequency in order to utilise the present invention to its fullest extent.

For oral administration in the form of a tablet or capsule, the vitamin D analogue and/or the pyrimidine nucleoside analogue may suitably be combined with an oral, non-toxic, pharmaceutically acceptable carrier such as ethanol, glycerol, water or the like. Furthermore, suitable binders, lubricants, disintegrating agents, flavouring agents and colourants may be added to the mixture, as appropriate. Suitable binders include, e.g., lactose, glucose, starch, gelatin, acacia gum, tragacanth gum, sodium alginate, carboxymethylcellulose, polyethylene glycol, waxes or the like. Lubricants include, e.g., sodium oleate, sodium stearate, magnesium stearate, sodium benzoate, sodium acetate, sodium chloride or the like. Disintegrating agents include, e.g., starch, methyl cellulose, agar, bentonite, xanthan gum or the like.

For the preparation of solid compositions such as tablets, the vitamin D analogue and/or pyrimidine nucleoside analogue are mixed with one or more excipients, such as the ones described above, and other pharmaceutical diluents such as water to make a solid preformulation composition containing a homogenous mixture of either or both active ingredients. The term “homogenous” is understood to mean that the active ingredient(s) is (are) dispersed evenly throughout the composition so that the composition may readily be subdivided into equally effective unit dosage forms such as tablets or capsules. The preformulation composition may then be subdivided into unit dosage forms containing from about 0.01 to about 100 μg of the vitamin D analogue and from about 100 to about 1000 mg of the pyrimidine nucleoside analogue.

Liquid formulations for either oral or parenteral administration of the active ingredient(s) include, e.g., aqueous solutions, syrups, aqueous or oil suspensions and emulsion with edible oils such as cottonseed oil, sesame oil, coconut oil or peanut oil. Suitable dispersing or suspending agents for aqueous suspensions include synthetic or natural gums such as tragacanth, alginate, acacia, dextran, sodium carboxymethylcellulose, gelatin, methylcellulose or polyvinylpyrolidone.

The pharmaceutical composition of the invention may also comprise either or both active ingredients dissolved in an appropriate, pharmaceutically acceptable solvent. Such compositions are primarily intended for parenteral administration.

For parenteral administration, e.g. intramuscular, intraperitoneal, subcutaneous or intravenous injection or infusion, the composition of the invention may include a sterile aqueous or non-aqueous solvent, in particular water, isotonic saline, isotonic glucose solution, buffer solution or other solvent conventionally used for parenteral administration of therapeutically active substances, in particular antiproliferative agents. The composition may be sterilised by, for instance, filtration through a bacteria-retaining filter, addition of a sterilising agent to the composition, irradiation of the composition, or heating the composition.

Alternatively, the active ingredient(s) may be provided as a sterile, solid preparation, e.g. a freeze-dried powder, which is dissolved in sterile solvent immediately prior to use.

The composition intended for parenteral administration may additionally comprise conventional additives such as stabilisers, buffers or preservatives, e.g. antioxidants such as methylhydroxybenzoate or the like.

In a further aspect, the invention relates to the use of a vitamin D analogue capable of upregulating the expression of cytidine deaminase in tumour cells in combination with a cytostatic pyrimidine nucleoside analogue for the preparation of a medicament for the treatment or amelioration of neoplastic diseases or conditions.

In a still further aspect, the invention relates to a method of treating or ameliorating neoplastic diseases or conditions, the method comprising administering, to a subject in need thereof, an effective amount of a vitamin D analogue capable of upregulating the expression of cytidine deaminase in tumour cells and, simultaneously or sequentially therewith, administering an effective amount of a pyrimidine nucleoside analogue.

In particular, neoplastic diseases or conditions to be treated by the present method include a number of solid tumours, including gastric cancer, intestinal or colorectal cancers, liver cancer, including metastases from colorectal cancer to the liver, ovarian cancer, breast cancer, head and neck cancer, endometrial cancer, pancreatic cancer etc. The present method further includes the use of the combination therapy as adjuvant treatment or for the initial management of metastatic disease.

In a preferred embodiment, the invention relates to a method of providing increased conversion of a fluoropyrimidine prodrug to 5-fluorouracil in a tumour cell, the method comprising contacting a tumour cell with an effective amount of a fluoropyrimidine prodrug and, simultaneously or sequentially therewith, contacting said tumour cell with an effective amount of a vitamin D analogue capable of upregulating the expression of cytidine deaminase in said cell.

As indicated above, it is preferred to administer the active ingredients in a dosage form intended for oral administration. As such, the active ingredients may be present in the same unit dosage form or may be provided in separate containers intended for simultaneous or sequential administration of the active ingredients. A suitable dosage of either active ingredient will depend, inter alia, on the age and condition of the patient, the severity of the disease to be treated and other factors well known to the practising physician. The compound may be administered either orally or parenterally according to different dosing schedules, e.g. continuously (e.g. daily over a period of time of a number of weeks), intermittently (e.g. in cycles of two weeks with one week of rest, in accordance with conventional practice in the administration of cytostatic agents) or with intervals such as, for instance, weekly intervals. In general, the dosage of the vitamin D analogue will be in the range of from about 0.01 to about 100 μg a day, such as 0.025-50 μg a day. The dosage of the pyrimidine nucleoside analogue may vary between wide limits dependent on the type of analogue to be administered, but is generally in the range of from about 100 to about 3500 mg/m²/day, such as about 300-2500 mg/m²/day, e.g. about 500-1500 mg/m²/day. The vitamin D analogue and/or pyrimidine nucleoside analogue used in the method of the invention may be administered once a day or in divided doses of two or more times a day, as appropriate.

In a still further aspect, the invention relates to a method of screening for vitamin D analogues with an increased activity in upregulating the expression of cytidine deaminase in tumour cells compared to the activity of 1α, 25-dihydroxyvitamin D₃, the method comprising (a) contacting tumour cells expressing cytidine deaminase with one or more test vitamin D analogues for a period of time sufficient for said analogues to exert an effect on the expression of cytidine deaminase in said cells, (b) determining the level of cytidine deaminase expression in cells treated with said analogues compared to the level of cytidine deaminase expression in untreated control cells, and (c) selecting vitamin D analogue(s) which, when in contact with said cells, result in upregulation of cytidine deaminase expression.

In the present screening method, 1α, 25-dihydroxyvitamin D₃ may preferably be used in step (b) as a positive control.

In step (c) of the present screening method, vitamin D analogues are preferably selected which exhibit an at least 50%, preferably at least 75%, e.g. at least 100%, higher cytidine deaminase upregulating activity in tumour cells compared to the activity of 1α, 25-dihydroxyvitamin D₃. An example of such a vitamin D analogue is seocalcitol.

The present screening method may also comprise the further step of selecting vitamin D analogue(s) which, when in contact with said cells, result in upregulation of both cytidine deaminase and thymidine phosphorylase expression. For this purpose, it is convenient to use either cultures of primary tumour cells expressing increased levels of thymidine phosphorylase or a transfected cell line expressing thymidine phosphorylase.

The invention is described in further detail in the following examples which are not in any way intended to limit the scope of the invention as claimed.

### EXAMPLES

### Example 1

HT-29 cells (a human colon cancer cell line) were grown in medium to which was added 0.0025% isopropanol as control, 10⁻⁷ M 1α, 25-dihydroxyvitamin D₃ or 10⁻⁷ M seocalcitol, respectively. The medium was changed on days 1, 4, 6, 8, 11 and 13, each time with addition of isopropanol, 1α, 25-dihydroxyvitamin D₃ or seocalcitol, as appropriate.

On each of the indicated days, cells from each culture were harvested, pelleted and frozen. For the cytidine deaminase and thymidine phosphorylase assays, the frozen cell pellet was thawed and resuspended in 150 μl of 0.1 M Tris-Hcl, pH 8.1. The cell suspension was sonicated for 2-5 seconds and centrifuged at 20000×g for 20 minutes at 4° C. 100 μl of cell extract was transferred to Eppendorf tubes containing 0.5 μl 1M DTT.

Cytidine Deaminase Assay

The cytidine deaminase activity in the cell extracts was determined by measuring the conversion of radiolabelled cytidine to cytidine and uracil. The assay is a combination of the methods described in J. Mejer and P. Nygaard, “Cytosine Arabinoside Phosphorylation and Deamination in Acute Myeloblastic Leukemia Cells”, Leukemia Research 2, 1978 pp. 127-131, and B. R. De Saint Vincent et al., “The Modulation of Thymidine Triphosphate Pool of Chinese Hamster Cells by dCMP Deaminase and UDP Reductase”, J. Biol. Chem. 255, 1980, pp. 162-167, which are hereby incorporated by reference.

The assay mixture has a total volume of 50 μl and the following composition:

5 μl 1M Tris-HCI, pH 7.2

5 μl 50 mM MgCl₂

5 μl 4 mM cytidine

4 μl water

2 μl ³H-cytidine (18.2 Ci/mmol, 1 mCi/ml)

25μl cell extract

The substrate and products were separated on PEI-cellulose plates placed in about 1.5 cm of a mixture of isopropanol:water:ethyl acetate 41:22:117.

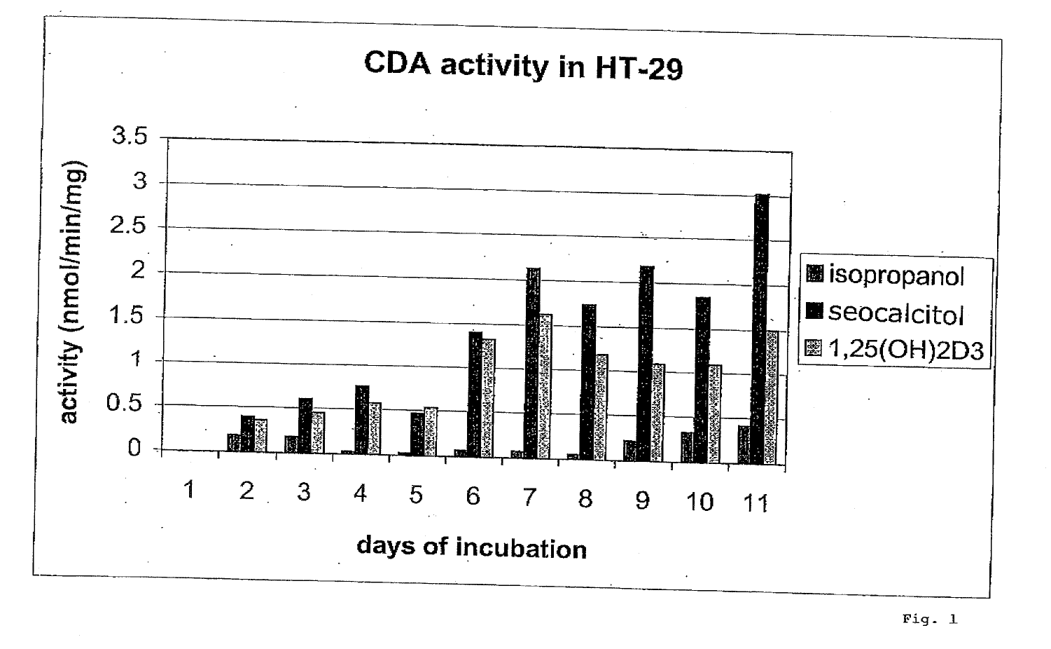

The results are shown in FIG. 1 from which it appears that for the first 9 days of incubation, the activity of cytidine deaminase in HT-29 cells treated with isopropanol was 0.08±0.059 nmol/min/mg. From day 9 to day 11, the activity increased to 0.42±0.069 nmol/min/mg. Incubation at day 1 for 24 hours with 10⁻⁷ M 1α,25-dihydroxyvitamin D₃ and seocalcitol resulted in a 4.5 and 4.9 fold increase, respectively, in cytidine deaminase activity. A maximum increase was observed after 8 days of incubation and was 15.5 fold with 1α,25-dihydroxyvitamin D₃ (p<0.0001). Incubation with seocalcitol resulted in an increase which was twice that observed for 1α, 25-dihydroxyvitamin D₃ (p<0.0001).

Thymidine Phosphorylase Assay

The thymidine phosphorylase activity was determined by measuring the conversion of radiolabelled thymidine to thymine. The assay method used was a slight modification of the method described in T. Sumizawa et al., “Thymidine Phosphorylase Activity Associated with Platelet-Derived Endothelial Cell Growth Factor”, J. Biochem. 114, 1993, pp. 9-14, which is hereby incorporated by reference.

The assay mixture had a total volume of 80 μl and the following composition:

10 μl 50 mM sodium phosphate, pH 7.0

10 μl mM Tris-HCl, pH 7.6

4 μl mM thymidine

4 μl water

2 μl ³H-thymidine (5 Ci/mmol, 1 mCi/ml)

50 μl cell extract

The substrate and product were separated on PEI-cellulose plates placed in about 1.5 cm of water.

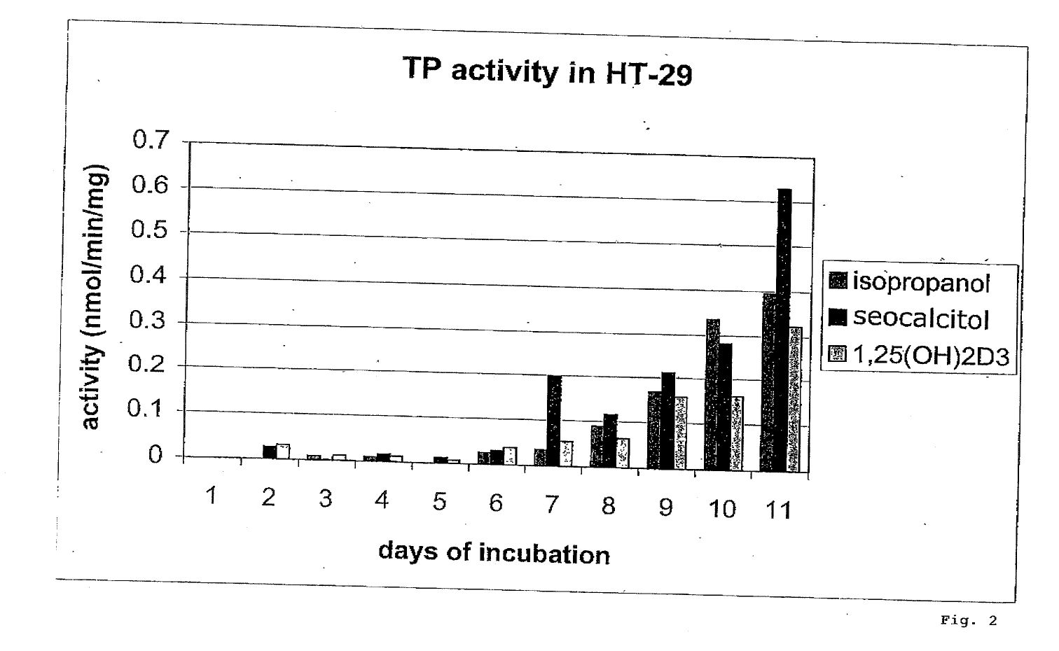

The results are shown in FIG. 2 from which it appears that thymidine phosphorylase activity was unchanged for the first 5 days of incubation of the isopropanol-treated HT-29 cells (0.014±0.009 nmol/min/mg). Then the activity increased to 0.395±0.062 nmol/min/mg at day 11. In the presence of 1α, 25-dihydroxyvitamin D₃ or seocalcitol, the thymidine phosphorylase activity was the same as in isopropanol-treated cells for the first 5 days.

Then, the increase in thymidine phosphorylase activity in 1α, 25-dihydroxyvitamin D₃ treated cells was significantly lower (0.323 nmol/min/mg at day 11, p<0.05) than in the control cells, whereas the increase in seocalcitol-treated cells was significantly higher (0.672 nmol/min/mg at day 11, p<0.0002).

### Example 2

The effect of administration of 1α, 25-dihydroxyvitamin D₃ or seocalcitol (EB 1089) on growth of FHC cells was investigated. FHC cells are normal human colon epithelial cells (ATCC CRL-1831) considered to be a good model of normal human colon epithelium. The cells were grown in a culture medium supplemented with 10⁻⁷ M 1α,25-dihydroxyvitamin D₃ or seocalcitol for 14 days.

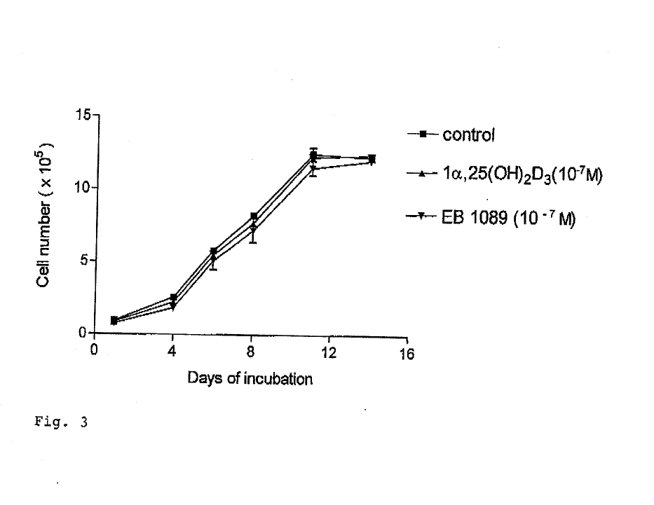

The results are shown in FIG. 3 from which it appears that the growth of the FHC cells is not significantly affected by the presence of 1α,25-dihydroxyvitamin D₃ or seocalcitol compared to the growth of untreated control cells.

Similarly, cytidine deaminase and thymidine phosphorylase activity in FHC cells following continuous exposure to 1α,25-dihydroxyvitamin D₃ or seocalcitol was determined as described in example 1. The cytidine deaminase and thymidine phosphorylase activity in control cells remained constant at 0.07±0.03 and 0.25±0.04 nmol/min/mg, respectively, over a period of 11 days and was unaffected by the presence of 1α,25-dihydroxyvitamin D₃ or seocalcitol.

### Example 3

The effect on the growth of HT-29 cells subjected to a combined treatment with seocalcitol and capecitabine is investigated.

In vivo, capecitabine is initially metabolised in the liver by a carboxylesterase to 5′-deoxy-5-fluorocytidine. The metabolite is then further converted by cytidine deaminase to 5′-deoxy-5-fluorouridine which, in turn, is converted to 5-FU by thymidine phosphorylase. As HT-29 cells lack the carboxylesterase, it is necessary initially to treat the capecitabine used in the experiment with a liver homogenate containing the post-mitochondrial liver fraction (S9) substantially as described in A-M Kissmeyer and IT Mortensen, Xenobiotica 30(8), 2000, pp. 815-830, to obtain enzymatic conversion thereof to 5′-deoxy-5-fluorocytidine. The compound is then isolated by preparative HPLC substantially as described by A-M Kissmeyer and JT Mortensen, supra.

Like many other established cancer cell lines (and unlike cultures of primary tumour cells), HT-29 expresses very low levels of thymidine phosphorylase. The effect of the combined treatment on HT-29 cells in terms of growth suppression is expected to be limited because a limited amount of 5′-deoxy-5-fluorouridine will actually be converted to the active 5-FU by this cell line. A cell line stably transfected with cDNA encoding human thymidine phosphorylase is therefore constructed by the following procedure.

Total RNA is isolated from HT-29 cells and the thymidine phosphorylase cDNA is RT-PCR amplified into one single fragment using the oligonucleotides 5′-CGA TGG CAG CCT TGA TGA CC-3′ (corresponding to nucleotides 122-141 of thymidine phosphorylase cDNA submitted by Finnis C. et al. (1991), Genbank Accession No. M 63193) and 5′-TGC CGG CAA AGG AGC TTl AT-3′ (corresponding to nucleotides 1569-1588 of thymidine phosphorylase cDNA submitted by Finnis C. et al. (1991), Genbank Accession No. M 63193). The PCR product is ligated into the pCR-Blunt-II TOPO vector (Invitrogen). A eucaryotic transfection vector is constructed by PCR amplification of the open reading frame encoding thymidine phosphorylase with the following primers 5′-CCC AAG CTT GCC ACC ATG GCA GCC TTG ATG ACC CCG-3′ and 5′-GCT CTA GAG CGG CAA AGG AGC TTT ATT GCT. The resulting PCR product is then ligated into the pcDNA3.1 (+) vector (Invitrogen) using the Hind III and Xba I sites thereby creating an eucaryotic expression vector designed pcDNA3.1/TP.

The pcDNA3.1/TP vector is introduced into HT-29 cells using liposome mediated transfection, and stably transfected HT-29 clones are selected using G418 (Invitrogen) containing medium. Ten G418 resistant clones are chosen for further evaluation and analyzed for expression of TP mRNA and activity. A HT-29/TP clone having a thymidine phosphorylase activity at levels comparable to those in primary tumour cells is chosen for further experiments.

The stably transfected HT-29/TP cells expressing thymidine phosphorylase are grown in the same way as parental HT-29 cells (cf. example 1) with the exception that G418 is included in the medium for maintaining the transfected phenotype.

The cells are exposed to a combination of seocalcitol and 5′-deoxy-5-fluorocytidine (isolated as described above) according to three different regimens:

(a) continuous exposure to both 10⁻⁷ M seocalcitol and 0.2 μM, 1 μM or 2 82 M 5′-deoxy-5-fluorocytidine for 11 days;

(b) continuous exposure to 10⁻⁷ M seocalcitol for 6 days followed by a single (bolus) exposure on day 6 to 2 μM 5′-deoxy-5-fluorocytidine; the effect on cell growth is measured on day 11;

(c) 2 hours of exposure to 10⁻⁷ M seocalcitol on day 1 (it has been found in a previous experiment that 2 hours of exposure to seocalcitol had an equivalent effect on cell growth and enzymatic activity as continuous exposure) followed by single (bolus) exposure to 2 μM 5′-deoxy-5-fluorocytidine on day 6; the effect on cell growth is measured on day 11.

The growth of the cells is determined by counting cells grown in T25 culture flasks (Nunc). After removing the medium and rinsing with 2 ml of a solution containing 0.25% trypsin and 0.03% EDTA, the solution is removed and an additional 1 ml trypsin-EDTA solution is added.

The flask is allowed to sit at 37° C. until the cell detach. The flask is shaken vigorously to make sure that all cell detach. Cells are removed by fresh medium. The cell suspension is passed through a needle if there is a tendency for the cells to cluster. 200 μl of the suspension is added to 10 ml 0.9% NaCl and counted in a Coulter Counter (COULTER® Particle Count and Size Analyzer Z₂, Beckman Coulter Inc., CA, USA). Viability is monitored by eosin exclusion.

### Example 4

In vivo experiments

In vivo experiments are conducted in a colorectal xenograft model as follows:

One experiment assessing the efficacy and toxicity of seocalcitol in 3 different doses versus vehicle. Seocalcitol is administered by oral gavage in doses ranging from 0.5-2.0 micrograms/kg/day.

One experiment assessing the efficacy and toxicity of capecitabine versus untreated controls. Capecitabine is administered as described in Biochem. Pharmacol. 55(7), 1998, pp. 1091-1097.

One experiment assessing the efficacy and toxicity of combined treatment with seocalcitol and capacitabine versus the individual treatments and controls. Seocalcitol and capecitabine are administered as above in doses identified in the previous experiments.

Each experiment includes 60 immunodeficient female NMRI nu/nu mice.

All mice are injected with 5×10⁶ HT-29 cells expressing thymidine phosphorylase (HT-29/TP cells prepared as described in example 3) in a volume of 0.2 mL subcutaneously in both flanks.

Tumours are left to grow for approximately 1 week before dosing is initiated.

When tumours are established, the mice are divided into 4-6 groups. Depending on the type of experiment, each group receives treatment for approximately 14 days with either the vehicle, seocalcitol, capecitabine or a combination of seocalcitol and capecitabine. Mice are sacrificed at the end of treatment or earlier in the event of very rapid tumour growth and/or ulceration of tumours.

All mice are weighed following injection of tumour cells, immediately before treatment, twice weekly during treatment and at the end of treatment.

Changes in tumour measurements and weight are analysed using an F-test (variance ratio test).

1. A pharmaceutical composition comprising, as a first active ingredient, a vitamin D analogue capable of upregulating the expression of cytidine deaminase in tumour cells and, as a second active ingredient, a cytostatic pyrimidine nucleoside analogue together with a pharmaceutically acceptable excipient or vehicle.

2. A composition according to claim 1, wherein the pyrimidine nucleoside analogue is a fluoropyrimidine prodrug.

3. A composition according to claim 2, wherein the fluoropyrimidine prodrug is selected from the group consisting of capecitabine and galocitabine.

4. A composition according to any of claims 1-3, wherein the vitamin D analogue is one which exhibits an at least 50%, preferably at least 75%, e.g. at least 100%, higher cytidine deaminase expression upregulating activity in tumour cells compared to the activity of 1α, 25-dihydroxyvitamin D₃.

5. A composition according to claim 2, wherein the vitamin D analogue is one which is capable of upregulating the expression of both cytidine deaminase and thymidine phosphorylase in tumour cells so as to effect increased levels of conversion of said fluoropyrimidine prodrug to 5-fluorouracil in tumour cells.

6. A composition according to any of claims 1-5, wherein the vitamin D analogue is a compound of formula I 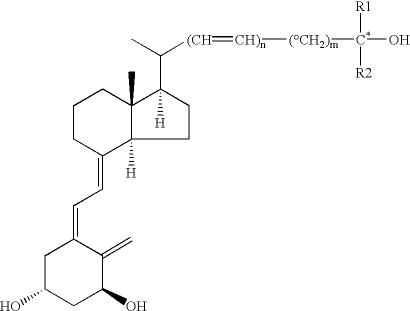
wherein p1 n is 2 or 3, m is 0 or an integer from 1 to 4;
R¹ and R², which are the same or different, are independently hydrogen or C₁₋₈ hydrocarbyl or, together with the carbon atom to which they are attached (marked with an asterisk in formula I), R¹ and R² form a saturated or unsaturated C₃₋₈ carbocyclic ring,
R¹ and/or R² and/or one of the m carbon atoms (marked with “^{o}” in formula I) being optionally substituted by one or more chloro or fluorine atoms or C₁₋₂ alkyl; or derivatives of compounds of formula I in which one or more hydroxy groups are transformed into —O-acyl or —O-glycosyl or phosphate ester groups, such masked groups being hydrolysable in vivo.

7. A composition according to claim 6, wherein the compound of formula I is seocalcitol.

8. A pharmaceutical combination composition comprising, in separate containers and intended for simultaneous or sequential administration, a vitamin D analogue capable of upregulating the expression of cytidine deaminase in tumour cells as a first active ingredient together with a pharmaceutically acceptable excipient or vehicle and a cytostatic pyrimidine nucleoside analogue as a second active ingredient together with a pharmaceutically acceptable excipient or vehicle.

9. A composition according to claim 8, wherein the pyrimidine nucleoside analogue is a fluoropyrimidine prodrug.

10. A composition according to claim 9, wherein the fluoropyrimidine prodrug is selected from the group consisting of capecitabine and galocitabine.

11. A composition according to any of claims 8-10, wherein the vitamin D analogue is one which exhibits an at least 50%, preferably at least 75%, e.g. at least 100%, higher cytidine deaminase expression upregulating activity in tumour cells compared to the activity of 1α, 25-dihydroxyvitamin D₃.

12. A composition according to claim 9, wherein the vitamin D analogue is one which is capable of upregulating the expression of both cytidine deaminase and thymidine phosphorylase in tumour cells so as to effect increased levels of conversion of said fluoropyrimidine prodrug to 5-fluorouracil in tumour cells.

13. A composition according to any of claims 8-12, wherein the vitamin D analogue is a compound of formula I 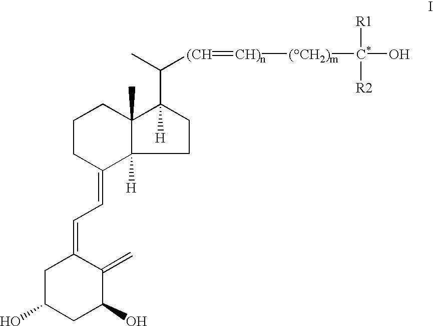
wherein
n is 2 or 3, m is 0 or an integer from 1 to 4;
R¹ and R², which are the same or different, are independently hydrogen or C₁₋₈ hydrocarbyl or, together with the carbon atom to which they are attached (marked with an asterisk in formula I), R¹ and R² form a saturated or unsaturated C₃₋₈ carbocyclic ring,
R¹ and/or R² and/or one of the m carbon atoms (marked with “^{o}” in formula I) being optionally substituted by one or more chloro or fluorine atoms or C₁₋₂ alkyl; or derivatives of compounds of formula I in which one or more hydroxy groups are transformed into —O-acyl or —O-glycosyl or phosphate ester groups, such masked groups being hydrolysable in vivo.

14. A composition according to claim 13, wherein the compound of formula I is seocalcitol.

15. Use of a vitamin D analogue capable of upregulating the expression of cytidine deaminase in tumour cells in combination with a cytostatic pyrimidine nucleoside analogue for the preparation of a medicament for the treatment or amelioration of neoplastic diseases or conditions.

16. The use of claim 15, wherein the pyrimidine nucleoside analogue is a fluoropyrimidine prodrug.

17. The use of claim 16, wherein the fluoropyrimidine prodrug is selected from the group consisting of capecitabine and galocitabine.

18. The use of any of claims 15-17, wherein the vitamin D analogue is one which exhibits an at least 50%, preferably at least 75%, e.g. at least 100%, higher cytidine deaminase expression upregulating activity in tumour cells compared to the activity of 1α, 25-dihydroxyvitamin D₃.

19. The use of claim 16, wherein the vitamin D analogue is one which is capable of upregulating the expression of both cytidine deaminase and thymidine phosphorylase in tumour cells so as to effect increased levels of conversion of said fluoropyrimidine prodrug to 5-fluorouracil in tumour cells.

20. The use of any of claims 15-19, wherein the vitamin D analogue is a compound of formula I 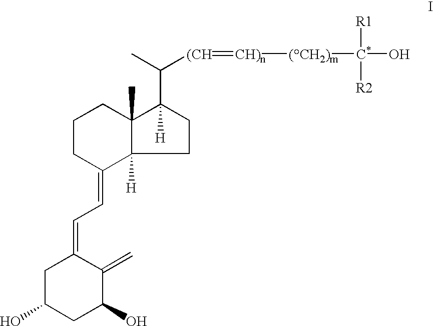
wherein
n is 2 or 3, m is 0 or an integer from 1 to 4;
R¹ and R², which are the same or different, are independently hydrogen or C₁-₈ hydrocarbyl or, together with the carbon atom to which they are attached (marked with an asterisk in formula I), R¹ and R² form a saturated or unsaturated C₃₋₈ carbocyclic ring,
R¹ and/or R² and/or one of the m carbon atoms (marked with “^{o}” in formula I) being optionally substituted by one or more chloro or fluorine atoms or C₁₋₂ alkyl; or derivatives of compounds of formula I in which one or more hydroxy groups are transformed into —O-acyl or —O-glycosyl or phosphate ester groups, such masked groups being hydrolysable in vivo.

21. The use of claim 20, wherein the compound of formula I is seocalcitol.

22. The use of claim 15, wherein the medicament comprises the vitamin D analogue and the pyrimidine nucleoside analogue in separate containers intended for simultaneous or sequential administration of the vitamin D analogue and the pyrimidine nucleoside analogue.

23. The use of claim 22, wherein the medicament comprises the vitamin D analogue and the pyrimidine nucleoside analogue in unit dosage form.

24. The use according to any of claims 15-23, wherein the neoplastic disease or condition is selected from the group consisting of gastrointestinal cancer, including colorectal cancer, liver cancer, breast cancer, pancreatic cancer, head and neck cancer, bladder cancer, ovarian cancer and endometrial cancer.

25. A method of treating or ameliorating neoplastic diseases or conditions, the method comprising administering, to a subject in need thereof, an effective amount of a vitamin D analogue capable of upregulating the expression of cytidine deaminase in tumour cells and, simultaneously or sequentially therewith, administering an effective amount of a pyrimidine nucleoside analogue.

26. The method of claim 25, wherein the pyrimidine nucleoside analogue is a fluoropyrimidine prodrug.

27. The method of claim 26, wherein the fluoropyrimidine prodrug is selected from the group consisting of capecitabine and galocitabine.

28. The method of any of claims 25-27, wherein the vitamin D analogue is one which exhibits an at least 50%, preferably at least 75%, e.g. at least 100%, higher cytidine deaminase expression upregulating activity in tumour cells compared to the activity of 1α, 25-dihydroxyvitamin D₃.

29. The method of claim 26, wherein the vitamin D analogue is one which is capable of upregulating the expression of both cytidine deaminase and thymidine phosphorylase in tumour cells so as to effect increased levels of conversion of said fluoropyrimidine prodrug to 5-fluorouracil in tumour cells.

30. The method of any of claims 25-29, wherein the vitamin D analogue is a compound of formula I 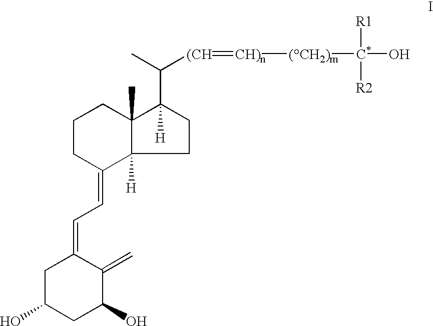
wherein
n is 2 or 3, m is 0 or an integer from 1 to 4;
R¹ and R², which are the same or different, are independently hydrogen or C₁₋₈ hydrocarbyl or, together with the carbon atom to which they are attached (marked with an asterisk in formula I), R¹ and R² form a saturated or unsaturated C₃₋₈ carbocyclic ring,
R¹ and/or R² and/or one of the m carbon atoms (marked with “^{o}” in formula I) being optionally substituted by one or more chloro or fluorine atoms or C₁₋₂ alkyl; or derivatives of compounds of formula I in which one or more hydroxy groups are transformed into —O-acyl or —O-glycosyl or phosphate ester groups, such masked groups being hydrolysable in vivo.

31. The method of claim 30, wherein the compound of formula I is seocalcitol.

32. A method of providing increased conversion of a fluoropyrimidine prodrug to 5-fluorouracil in a tumour cell, the method comprising contacting a tumour cell with an effective amount of a fluoropyrimidine prodrug and, simultaneously or sequentially therewith, contacting said tumour cell with an effective amount of a vitamin D analogue capable of upregulating the expression of cytidine deaminase in said cell.

33. The method of claim 32, wherein the vitamin D analogue is one which exhibits an at least 50%, preferably at least 75%, e.g. at least 100%, higher cytidine deaminase expression upregulating activity in tumour cells compared to the activity of 1α, 25-dihydroxyvitamin D₃.

34. The method of claim 32, wherein the vitamin D analogue is one which is capable of upregulating the expression of both cytidine deaminase and thymidine phosphorylase in tumour cells.

35. The method of any of claims 32-34, wherein the vitamin D analogue is a compound of formula I 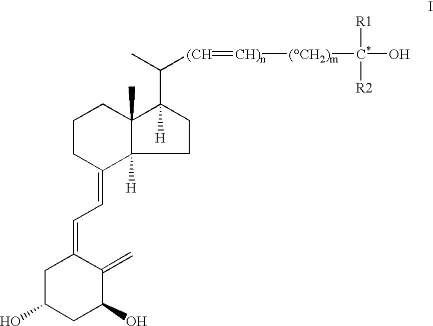
wherein
n is 2 or 3, m is 0 or an integer from 1 to 4;
R¹ and R², which are the same or different, are independently hydrogen or C₁₋₈ hydrocarbyl or, together with the carbon atom to which they are attached (marked with an steris in formula I), R¹ and R² form a saturated or unsaturated C₃₋₈ carbocyclic ring,
R¹ and/or R² and/or one of the m carbon atoms (marked with “^{o}” in formula I) being optionally substituted by one or more chloro or fluorine atoms or C₁₋₂ alkyl; or derivatives of compounds of formula I in which one or more hydroxy groups are transformed into —O-acyl or —O-glycosyl or phosphate ester groups, such masked groups being hydrolysable in vivo.

36. The method of claim 35, wherein the compound of formula I is seocalcitol.

37. The method of any of claims 32-36, wherein the fluoropyrimidine prodrug is selected from the group consisting of capecitabine and galocitabine.

38. A method of screening for vitamin D analogues with an increased activity in upregulating the expression of cytidine deaminase in tumour cells compared to the activity of 1α, 25-dihydroxyvitamin D₃, the method comprising (a) contacting tumour cells expressing cytidine deaminase with one or more test vitamin D analogues for a period of time sufficient for said analogues to exert an effect on the expression of cytidine deaminase in said cells, (b) determining the level of cytidine deaminase expression in cells treated with said analogues compared to the level of cytidine deaminase expression in untreated control cells, and (c) selecting vitamin D analogue(s) which, when in contact with said cells, result in upregulation of cytidine deaminase expression.

39. The method of claim 38, wherein 1α, 25-dihydroxyvitamin D₃ is additionally used in step (b) as a positive control.

40. The method of claim 38 or 39, wherein, in step (c), vitamin D analogues are selected which exhibit an at least 50%, preferably at least 75%, e.g. at least 100%, higher cytidine deaminase upregulating activity in tumour cells compared to the activity of 1α, 25-dihydroxyvitamin D₃.

41. The method of claim 40, wherein the vitamin D analogue selected in step (c) is seocalcitol.

42. The method of any of claims 38-41 comprising the further step of selecting vitamin D analogue(s) which, when in contact with said cells, result in upregulation of both cytidine deaminase and thymidine phosphorylase expression.
